(19) United States
(12) Patent Application Publication
(10) Pub. No.: US 2026/0199711 A1
(43) Pub. Date: Jul. 16, 2026

(54) POSTOPERATIVE LYMPHATIC TREATMENT SYSTEM USING ULTRASOUND AND MANUAL DRAINAGE

(71) Applicant: Eva Claiborne, Tiburon, CA (US)

(72) Inventor: Eva Claiborne, Tiburon, CA (US)

(21) Appl. No.: 19/560,004

(22) Filed: Mar. 7, 2026

(51) Int. Cl. A61N 7/00

(52) U.S. Cl. CPC A61N 7/00

(57) ABSTRACT

The Ultrasound Lymphatic Treatment System and associated Manual and Ultrasound-Enhanced Lymphatic Drainage Method provide an integrated solution for preoperative and postoperative lymphatic stimulation. The system comprises a programmable ultrasound delivery subsystem, manual drainage implements, and a protocol-driven control module with real-time sensor feedback. The method includes individualized patient evaluation, mapping of lymphatic zones, and sequential application of manual drainage and low-frequency ultrasound along anatomically validated pathways. The system generates a patient-specific lymphatic zone map and session ultrasound parameter set, enabling reproducible, zone-targeted therapy. Closed-loop regulation of ultrasound parameters and contact pressure ensures safety and efficacy. The invention standardizes the combination of manual and ultrasound modalities, accelerates edema resolution, reduces pain, and supports immune recovery, addressing the lack of coordinated, evidence-based lymphatic care in surgical patients.

### TECHNICAL FIELD

The invention relates generally to the field of lymphatic therapy and ultrasound medical devices and more specifically to a new and useful postoperative lymphatic treatment system using ultrasound and manual drainage in the field of lymphatic therapy and ultrasound medical devices.

### BACKGROUND

Surgical procedures, whether elective or medically necessary, impose significant physiological stress on the body, particularly affecting the lymphatic system responsible for fluid balance, waste removal, and immune function. Prior to the development of comprehensive perioperative lymphatic care protocols, preoperative preparation of patients focused primarily on cardiovascular health, nutritional status, and infection prevention. Conventional preoperative protocols failed to address the lymphatic system as a target for preparation, leaving the skin and immune system in a suboptimal state before surgical intervention. Medical practitioners recognized that patients entering surgery with compromised or unstimulated lymphatic function experienced greater postoperative complications, yet no systematic approach existed to activate and prepare the lymphatic system in anticipation of surgical trauma.

Following surgical procedures, patients commonly experience prolonged swelling, bruising, and pain that extend recovery timelines and diminish quality of life during the healing period. Traditional postoperative care relied heavily on compression garments, elevation, and pharmacological interventions to manage these symptoms. Manual lymphatic drainage performed by trained therapists provided some benefit, but access to such specialized care remained limited and inconsistent. Existing postoperative lymphatic care approaches failed to adequately address the underlying stagnation of lymphatic fluid that contributed to persistent edema and inflammatory responses. The absence of effective lymphatic stimulation methods during the critical postoperative window resulted in suboptimal healing outcomes for many surgical patients.

The therapeutic potential of combining manual lymphatic massage techniques with ultrasound technology remained largely unexplored in clinical practice. Manual lymphatic drainage, while beneficial when performed correctly, produced variable results depending on practitioner skill and patient compliance. Therapeutic ultrasound devices existed independently for various musculoskeletal applications, but integration of ultrasound energy with lymphatic massage protocols had not been systematically developed or validated. Practitioners lacked guidance on how to sequence, combine, or coordinate manual techniques with ultrasound application to achieve synergistic effects on lymphatic drainage and tissue healing.

Ultrasound technology has been employed in medical and therapeutic contexts for decades, yet standardized parameters for lymphatic system activation remained undefined. Existing ultrasound applications focused on imaging, deep tissue heating, or targeted tissue destruction rather than lymphatic stimulation. The frequency ranges, power densities, treatment durations, and application patterns optimal for enhancing lymphatic flow had not been established through systematic investigation. Practitioners utilizing ultrasound for general therapeutic purposes applied inconsistent parameters without specific protocols designed to maximize lymphatic system response.

Post-surgical recovery depends significantly on the restoration of normal immune and metabolic function, processes intimately connected to lymphatic circulation. Stagnant lymph flow following surgery impairs the clearance of cellular debris, inflammatory mediators, and metabolic waste products from surgical sites. Conventional recovery protocols addressed wound healing and pain management but neglected the role of active lymphatic circulation in supporting immune surveillance and metabolic normalization. Patients with compromised lymphatic flow experienced delayed tissue regeneration, increased susceptibility to infection, and prolonged inflammatory states that hindered return to normal function.

### SUMMARY OF THE INVENTION

The present invention relates to an Ultrasound Lymphatic Treatment System and an associated Manual and Ultrasound-Enhanced Lymphatic Drainage Method for producing a Patient-Specific Lymphatic Zone Map. The Ultrasound Lymphatic Treatment System integrates an ultrasound delivery subsystem, a manual lymphatic drainage subsystem, a control and feedback module, a sensor suite, and supporting components including a patient positioning and applicator mount, a sterile coupling medium cartridge, and a power supply module. The Manual and Ultrasound-Enhanced Lymphatic Drainage Method employs a structured sequence of patient evaluation, proximal node clearance, distal-to-proximal manual drainage, ultrasound parameter configuration, ultrasound application along lymphatic pathways, alternating manual-ultrasound cycles, post-treatment assessment, and session scheduling to achieve comprehensive lymphatic system activation and tissue decongestion.

The Ultrasound Lymphatic Treatment System addresses the lack of preoperative lymphatic stimulation methods by providing a patient evaluation and lymphatic zone mapping step that generates a Patient-Specific Lymphatic Zone Map prior to any surgical intervention. The control and feedback module executes the patient evaluation and lymphatic zone mapping step, utilizing the sensor suite comprising a tissue impedance sensor, a contact pressure sensor, and a skin temperature sensor to assess baseline lymphatic function and tissue condition. The Patient-Specific Lymphatic Zone Map serves as an input to subsequent proximal node clearance, distal-to-proximal manual drainage sequence, ultrasound parameter configuration, and ultrasound application along lymphatic pathways steps, thereby enabling targeted preoperative preparation of the skin and immune system. Prior art systems lack integrated mapping capabilities that correlate anatomical lymphatic zones with individualized treatment parameters, whereas the present invention produces a zone map that directly informs both manual and ultrasound-based interventions.

The Ultrasound Lymphatic Treatment System resolves insufficient postoperative lymphatic care by implementing a comprehensive post-treatment assessment and documentation step that quantifies treatment outcomes and guides ongoing care. The post-treatment assessment and documentation step includes edema quantification and pain and mobility scoring substeps, each producing respective datasets that inform session scheduling and patient education. The control and feedback module stores post-treatment outcome datasets in the data logging memory and transmits the datasets through the connectivity interface for longitudinal tracking. The session scheduling and patient education step outputs a patient self-care schedule and instruction packet, ensuring continuity of care beyond clinical sessions. Compared to prior art approaches that provide only episodic treatment without systematic outcome tracking, the present invention establishes a closed-loop feedback mechanism that adapts subsequent sessions based on documented patient progress, thereby reducing prolonged swelling, bruising, and pain.

The Manual and Ultrasound-Enhanced Lymphatic Drainage Method addresses the absence of protocols combining manual lymphatic massage with therapeutic ultrasound by establishing an alternating manual-ultrasound cycles step that interleaves manual drainage with ultrasound application. The manual lymphatic drainage subsystem executes proximal node clearance and distal-to-proximal manual drainage sequence steps using a massage implement having a contoured massage surface and a pressure sensor array. The proximal node clearance step results in a proximal nodes decongested configuration that enables subsequent ultrasound application along lymphatic pathways and alternating manual-ultrasound cycles steps. The distal-to-proximal manual drainage sequence includes upper body manual stroke sequence and lower body manual stroke sequence substeps, each resulting in a pre-drained tissue state that enables static nodal activation and dynamic vessel sweeping. The ultrasound delivery subsystem executes ultrasound application along lymphatic pathways through static nodal activation and dynamic vessel sweeping substeps, with the calibrated ultrasound parameter set and applied coupling medium layer serving as inputs. Prior art methods employ either manual drainage or ultrasound in isolation, whereas the present invention synergistically combines both modalities in a defined alternating sequence to achieve enhanced lymphatic drainage beyond what either modality achieves independently.

The Ultrasound Lymphatic Treatment System establishes a standardized ultrasound frequency and application regimen through the ultrasound parameter configuration step comprising frequency selection, duty cycle and intensity setting, and coupling medium application substeps. The generator electronics include a frequency synthesizer and a power amplifier stage that receive configuration from the ultrasound system parameterized state resulting from the frequency selection and duty cycle and intensity setting substeps. The frequency selection substep and the duty cycle and intensity setting substep each output an ultrasound parameter set that configures the ultrasound delivery subsystem, the generator electronics, the frequency synthesizer, the power amplifier stage, and the transducer applicator. The transducer applicator comprises a piezoelectric element stack, an acoustic matching layer, a coupling interface, and an integrated temperature sensor, with the coupling interface receiving configuration from the coupling layer present on skin state. The home-use postoperative lymphatic therapy variant and the breast-centric postoperative protocol variant each affect the ultrasound parameter set and the ultrasound system parameterized state, enabling protocol-specific parameter standardization. Prior art ultrasound systems lack defined parameter sets correlated to lymphatic activation outcomes, whereas the present invention provides reproducible frequency ranges, duty cycle ranges, and intensity ranges validated for lymphatic system stimulation.

The Manual and Ultrasound-Enhanced Lymphatic Drainage Method improves post-surgical immune and metabolic recovery by achieving a post-therapy decongested tissue state through the coordinated execution of manual and ultrasound interventions. The proximal nodes cleared state enables the manual and ultrasound-enhanced lymphatic drainage method to proceed with distal tissue mobilization, while the distal tissues primed state enables ultrasound application along lymphatic pathways. The ultrasound session settings loaded state configures the ultrasound delivery subsystem and enables the manual and ultrasound-enhanced lymphatic drainage method to deliver calibrated acoustic energy to lymphatic structures. The static nodal activation substep delivers focused ultrasound energy to lymph node regions, while the dynamic vessel sweeping substep applies ultrasound along lymphatic vessel pathways to promote fluid movement. The sensor suite monitors tissue impedance, contact pressure, and skin temperature throughout treatment, with the control and feedback module adjusting parameters based on sensor feedback. The cooling mechanism within the ultrasound delivery subsystem maintains tissue temperature within safe ranges during extended ultrasound application. Prior art systems fail to address stagnant lymph flow comprehensively, whereas the present invention restores lymphatic circulation through a multi-modal approach that activates both nodal and vessel components of the lymphatic system, thereby accelerating immune cell trafficking and metabolic waste clearance following surgical procedures.

### BRIEF DESCRIPTION OF FIGURES

FIG. 1 is a schematic representation of a lymph system

FIG. 2A is a schematic representation of relevant areas to a post operative treatment for an abdominoplasty procedure.

FIG. 2B is a schematic representation of relevant areas to a post operative treatment for an abdominoplasty procedure.

FIG. 2C is a schematic representation of relevant areas to a post operative treatment for an abdominoplasty procedure.

FIG. 3 depicts breast components relevant to post operative treatment after a breast augmentation procedure.

FIG. 4A depicts directions of motion over a face and back relevant to lymphatic treatment after face, neck or back surgery.

FIG. 4B depicts directions of motion over a back relevant to lymphatic treatment after face, neck or back surgery.

FIG. 5A depicts front side anatomy relevant to lymphatic treatment after liposuction

FIG. 5B depicts back side anatomy relevant to lymphatic treatment after liposuction,

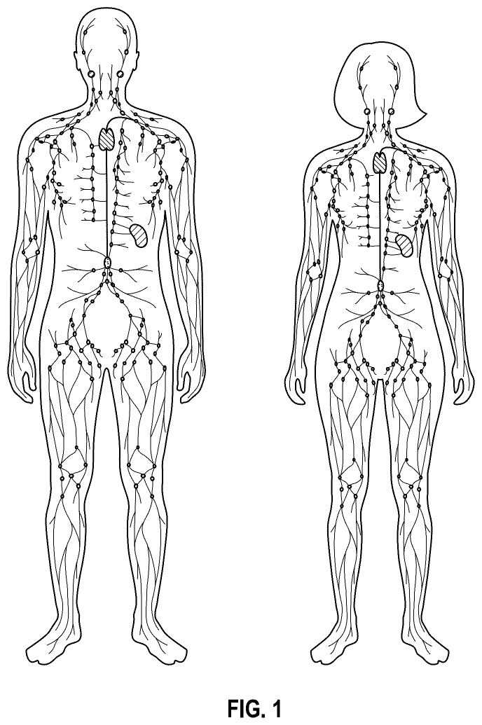

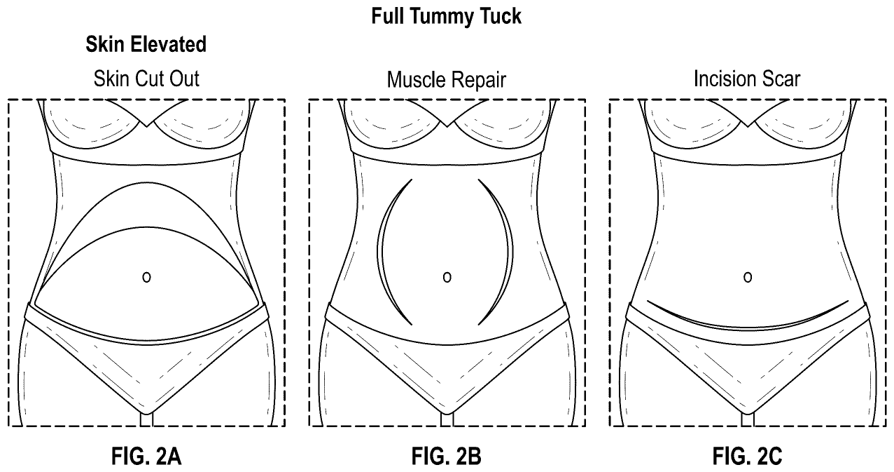

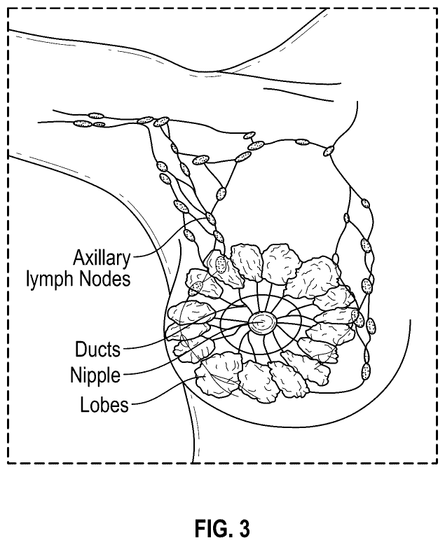

### DETAILED DESCRIPTION

### Ultrasound Lymphatic Treatment System

The ultrasound lymphatic treatment system can comprise an integrated apparatus engineered to deliver both preoperative and postoperative lymphatic stimulation by coordinating therapeutic ultrasound and manual drainage techniques. The system can include a therapeutic ultrasound delivery subsystem, which can incorporate a transducer capable of emitting acoustic energy at frequencies and intensities optimized for lymphatic vessel and nodal stimulation, generally within a range of 0.5 to 3.0 MHz and 0.1 to 2.0 W/cm² . The ultrasound subsystem can be configured to deliver energy in either pulsed or continuous mode, with programmable duty cycles and treatment durations that can be tailored to specific anatomical zones and individual patient requirements. This flexibility allows the system to address the technical challenge of providing standardized yet customizable lymphatic stimulation protocols, which is not addressed by conventional devices limited to fixed or non-optimized regimens.

In addition to the ultrasound delivery subsystem, the ultrasound lymphatic treatment system can further incorporate manual drainage implements, such as ergonomically contoured massage applicators or gloves. These implements can be used in conjunction with, or independently from, the ultrasound transducer to facilitate mechanical manipulation of the skin and subcutaneous tissue along established lymphatic drainage pathways. The system can define these pathways based on anatomical mapping of nodal basins and vessel trajectories, ensuring that both manual and acoustic modalities are applied along physiologically validated routes. This integration of manual and ultrasound modalities within a single platform addresses the technical challenge of combining mechanical and acoustic lymphatic stimulation in a reproducible and operator-independent manner, which is not achieved by prior art relying solely on manual massage or generic ultrasound application.

A protocol-driven control module can govern the sequencing, timing, and intensity of both ultrasound and manual modalities. The control module can include a microprocessor, a user interface, and programmable memory for storing standardized or customizable treatment regimens. The system can be equipped with patient-mount hardware, such as adjustable straps, harnesses, or docking stations, to ensure stable and reproducible applicator placement over targeted treatment zones. This arrangement can facilitate hands-free operation, reduce operator fatigue, and enable consistent application of therapy across multiple sessions and users, thereby addressing the challenge of inter-operator variability and inconsistent treatment delivery.

Integrated sensors, including but not limited to temperature, pressure, and contact sensors, can provide real-time feedback to the control module. The control module can utilize this feedback to enable closed-loop adjustment of ultrasound output and mechanical force, maintaining safe and effective treatment parameters throughout the session. The system can utilize consumable coupling media, such as medical-grade ultrasound gel, to ensure efficient acoustic transmission and minimize impedance at the skin interface. A power supply, which may be mains-powered or battery-operated, can support both portable and stationary operation, expanding the system's applicability to a range of clinical and home-care environments. These features collectively address the technical challenges of maintaining safe, effective, and reproducible lymphatic stimulation in diverse postoperative care settings.

The ultrasound lymphatic treatment system can be configured to execute zone-specific treatment protocols, wherein acoustic and manual stimuli are applied in a coordinated sequence to anatomical regions identified as prone to postoperative lymphatic congestion, such as the abdomen, breast, extremities, and cervical regions. The system can store and execute pre-programmed regimens that specify the order, duration, and intensity of treatment for each zone, with optional user override for individualized therapy. By integrating therapeutic ultrasound and manual drainage within a single, feedback-enabled platform, the system enables synergistic enhancement of lymphatic flow, reduction of interstitial edema, and acceleration of immune and metabolic recovery following surgical procedures. The system's architecture can support both standardized and patient-specific protocols, facilitating reproducible outcomes and improved postoperative care relative to conventional devices that lack such multimodal coordination. This comprehensive approach directly addresses the technical challenges of insufficient postoperative lymphatic care, lack of standardized protocols, and poor immune and metabolic recovery due to stagnant lymph flow.

### Ultrasound Delivery Subsystem

Generally, the ultrasound delivery subsystem can be configured to generate and transmit acoustic energy for the purpose of stimulating lymphatic flow in biological tissue. The ultrasound delivery subsystem can operate within a frequency range (e.g., between approximately 20 kHz and 100 kHz) suitable for activating lymphatic vessels and/or nodal regions. In one implementation, the ultrasound delivery subsystem can include generator electronics configured to modulate and supply electrical power, a transducer applicator configured to convert electrical energy into acoustic energy and couple the acoustic energy into skin and subcutaneous layers, and/or a cooling mechanism configured to maintain safe operating temperatures during treatment sessions. The ultrasound delivery subsystem can further include user interface controls and/or safety interlocks to enable adjustment of output parameters and/or to prevent operation outside of specified conditions. Thus, by providing controlled acoustic energy delivery along predetermined lymphatic pathways, the ultrasound delivery subsystem can address the absence of standardized ultrasound frequency and application regimens for lymphatic system activation while enabling enhanced lymphatic drainage when combined with manual techniques.

### Generator Electronics

Generally, the generator electronics can comprise a solid-state circuit assembly configured to synthesize programmable electrical waveforms suitable for driving the transducer applicator. The generator electronics can include a microcontroller and/or a digital signal processor that enables user selection and/or automated adjustment of output parameters, including frequency, amplitude, and/or duty cycle. In one implementation, the frequency can be adjustable within a range (e.g., between approximately 0.5 MHz and approximately 3.0 MHz). The generator electronics can be configured to deliver continuous and/or pulsed waveforms with output power adjustable across a range (e.g., from approximately 5 watts to approximately 100 watts), thereby allowing modulation of acoustic intensity according to treatment requirements and/or patient tissue characteristics. Additionally, the generator electronics can include an integrated impedance monitoring subsystem configured to continuously measure reflected electrical impedance at the transducer-tissue interface and dynamically adjust output parameters to maintain efficient acoustic coupling and/or minimize energy loss. The generator electronics can support programmable frequency sweeps and/or duty-cycle modulation, which can be tailored to target specific tissue depths and/or lymphatic structures. Further, the generator electronics can include safety interlocks, overcurrent protection, and/or user interface elements for real-time feedback and/or protocol customization. The generator electronics can support manual and/or automated operation modes, thereby enabling integration into standardized lymphatic drainage protocols that combine manual massage with therapeutic ultrasound application.

### Frequency Synthesizer

The frequency synthesizer can function as a core electronic component within the Generator Electronics of the Ultrasound Delivery Subsystem, which is itself a part of the broader Ultrasound Lymphatic Treatment System. The frequency synthesizer can generate electrical signals with precisely controlled frequencies, which are essential for driving therapeutic ultrasound transducers used in lymphatic stimulation protocols. In one implementation, the frequency synthesizer can incorporate a direct digital synthesis (DDS) core, which enables the generation of continuous sine wave, frequency-swept, or pulsed output waveforms. The DDS core can provide a selectable output frequency in the range of 20 kHz to 1 MHz, with a minimum frequency resolution of 1 Hz, thereby allowing for fine adjustment of ultrasound parameters to match the requirements of various lymphatic drainage protocols and anatomical treatment zones. The frequency synthesizer can be programmed to deliver single-frequency, multi-frequency, or time-varying frequency regimens, supporting both standardized and patient-specific treatment protocols. The output waveform type and frequency can be selected via a user interface or pre-programmed protocol, enabling adaptation to different tissue depths, lymphatic vessel sizes, and postoperative conditions. The frequency synthesizer can interface directly with the ultrasound transducer driver circuitry, providing the excitation signal that determines the acoustic output characteristics of the therapeutic ultrasound applicator. In some implementations, the frequency synthesizer can include digital or analog control inputs for integration with automated or semi-automated treatment systems, and can support feedback-based adjustment of frequency parameters in response to real-time tissue impedance or other physiological measurements. By enabling precise, programmable, and adaptive frequency control, the frequency synthesizer addresses the technical challenge of delivering reproducible and anatomically optimized ultrasound energy for enhanced lymphatic drainage, which is not achievable with conventional fixed-frequency or analog-tuned ultrasound generators.

### Transducer Applicator

The transducer applicator can serve as the primary interface for delivering therapeutic ultrasound energy to anatomical regions identified for lymphatic stimulation within the Ultrasound Lymphatic Treatment System. The transducer applicator can comprise a housing that is either handheld or mountable, providing flexibility for both clinician-directed and automated operation. Within the housing, a piezoelectric transducer stack can generate acoustic waves in the frequency range of approximately 0.5 to 3.0 MHz, which is suitable for activating lymphatic vessels and nodal basins at varying tissue depths. An acoustic matching layer can be positioned between the piezoelectric stack and the applicator head to optimize the transmission of ultrasound energy into biological tissue, thereby minimizing reflection losses at the tissue interface and ensuring efficient energy delivery to subcutaneous lymphatic structures.

The transducer applicator can further incorporate integrated sensors, such as temperature sensors, contact pressure sensors, and impedance sensors, which can continuously monitor and regulate treatment parameters in real time. These sensors can provide feedback to the control module, enabling closed-loop adjustment of ultrasound output and ensuring that the applied energy remains within safe and effective therapeutic windows. The housing of the transducer applicator can be ergonomically contoured to conform to various body regions, including the torso, limbs, and facial zones, which facilitates consistent acoustic coupling and maneuverability during treatment. In one implementation, the applicator can include detachable or interchangeable heads, allowing for sterile change-out between patients or adaptation to different anatomical sites, thereby supporting infection control and protocol flexibility.

In addition, the transducer applicator can feature visual or tactile indicators, such as LEDs or haptic actuators, to guide the operator in maintaining appropriate contact pressure and movement along prescribed lymphatic pathways. These indicators can be synchronized with the treatment protocol software, providing real-time prompts that standardize operator technique and reduce inter-user variability. The design of the transducer applicator can support both manual and automated operation, enabling seamless integration into protocols that combine manual lymphatic drainage with ultrasound-assisted therapy. By enabling reproducible placement, pressure, and movement patterns as specified in the lymphatic drainage protocol, the transducer applicator addresses the technical challenge of standardizing lymphatic stimulation across different operators, anatomical regions, and clinical settings, thereby improving the efficacy and safety of postoperative lymphatic care.

### Cooling Mechanism

Generally, the ultrasound delivery subsystem can include a cooling mechanism configured to dissipate heat generated by the generator electronics and/or the transducer applicator during operation. In one implementation, the cooling mechanism comprises a passive heat sink fabricated from thermally conductive materials, such as aluminum or copper, and can be thermally coupled to the power amplifier stage and/or the piezoelectric element stack. The heat sink can include a finned geometry configured to maximize surface area for convective and/or radiative heat transfer to the ambient environment. Additionally or alternatively, the cooling mechanism can include a micro-fan to provide forced-air cooling, thereby increasing the rate of heat dissipation from the heat sink. The cooling mechanism can be configured to maintain operational temperatures of the coupled components below a target threshold (e.g., less than 50° C.) during extended treatment sessions (e.g., exceeding 30 minutes of continuous operation). In one embodiment, thermal interface materials can be disposed between electronic components and the heat sink to enhance heat transfer efficiency. The cooling mechanism can be implemented as a passive-only configuration or as a hybrid passive-active configuration depending on anticipated thermal loads and/or duty cycle requirements of the ultrasound lymphatic treatment system. Further, the cooling mechanism can be arranged so as not to interfere with ergonomic handling of the transducer applicator or acoustic coupling at the coupling interface during manual and ultrasound-enhanced lymphatic drainage procedures.

### Manual Lymphatic Drainage Subsystem

The manual lymphatic drainage subsystem can provide mechanical stimulation to the lymphatic system by employing specialized manual techniques that follow anatomically defined lymphatic pathways. The subsystem can include massage implements and/or manual manipulation protocols, each designed to target both superficial and deep lymphatic vessels, nodal basins, and mapped drainage routes throughout the human body. The subsystem can be operated by a trained practitioner or by a user following a standardized protocol, with mechanical stimulation delivered through a sequence of strokes, compressions, or circular motions oriented along vectors that correspond to the direction of lymphatic flow toward regional lymph nodes, such as the cervical, axillary, inguinal, and popliteal nodes. The manual lymphatic drainage subsystem can function independently or in conjunction with a therapeutic ultrasound subsystem, allowing for alternating or combined manual and acoustic stimulation within a single treatment session. The subsystem can further allow for adjustment of pressure, stroke length, and frequency of application, with these parameters specified according to patient-specific factors such as tissue compliance, surgical site, and degree of postoperative edema. In scenarios where acoustic energy is contraindicated, such as in the presence of certain implants, open wounds, or patient intolerance, the subsystem can operate as a stand-alone modality to promote lymphatic drainage, reduce interstitial fluid accumulation, and facilitate immune and metabolic recovery. The subsystem can include implements such as soft-tipped rollers, silicone cups, or gloved hands, with the selection based on the anatomical region and desired depth of tissue engagement. The protocol can specify the duration of each manual drainage session, the sequence of treated regions, and the integration with other postoperative care steps. By standardizing and optimizing the delivery of manual lymphatic drainage in the context of postoperative recovery, the subsystem addresses the technical challenge of providing reproducible and anatomically targeted stimulation, thereby supporting the overall therapeutic objectives of the Manual and Ultrasound-Enhanced Lymphatic Drainage Method.

### Massage Implement

The massage implement can be configured for use in manual lymphatic drainage procedures and can comprise a contoured body or glove fabricated from biocompatible materials, such as medical-grade silicone, thermoplastic elastomer, or woven synthetic fabric. The massage implement can be designed to conform to the anatomical curvature of the treatment region, thereby enabling uniform distribution of light pressure across the skin surface. In one implementation, the massage implement can incorporate surface texturing, such as ridges or nodules, which can enhance mechanical stimulation of superficial lymphatic vessels. The device can be dimensioned to allow the application of pressure not exceeding 40 mmHg, as measured by an in-line manometer or pressure-sensing film, to avoid collapse of lymphatic capillaries while promoting centripetal movement of interstitial fluid toward regional lymph node basins. The massage implement can be provided in multiple sizes or shapes to accommodate different body regions, including but not limited to the neck, trunk, extremities, and face. In some embodiments, the massage implement can include integrated sensors to monitor applied pressure or track stroke direction and duration. The device can be used in conjunction with a therapeutic ultrasound applicator, either sequentially or simultaneously, to enhance lymphatic flow as part of a combined manual and ultrasound-assisted drainage protocol. The massage implement can be sterilizable or single-use, and may be provided with a lubricating agent to reduce friction and facilitate gliding over the skin. By standardizing manual lymphatic drainage techniques, the massage implement can reduce operator fatigue and ensure reproducible application of pressure and stroke geometry across treatment sessions. This configuration addresses the technical challenge of inconsistent manual lymphatic drainage by providing a device that enables uniform, safe, and effective stimulation of lymphatic flow, thereby improving postoperative recovery and reducing the risk of tissue trauma or inadequate drainage.

### Contoured Massage Surface

The contoured massage surface can be formed from a thermoplastic elastomer or a comparable biocompatible polymer, and is engineered to provide a smooth, continuous, and anatomically conforming interface for contact with human skin during lymphatic drainage procedures. The contoured massage surface can be shaped to match the curvature of specific body regions, such as the abdomen, limbs, or neck, with the geometry including convex, concave, or compound curves and radii of curvature selected to correspond to typical anthropometric measurements for the intended treatment area. In one implementation, the contoured massage surface can be integrated into a handheld applicator or an ultrasound transducer housing, allowing the applicator to glide along the skin without causing localized compression, pinching, or shear forces that could disrupt lymphatic flow or damage subcutaneous tissues. The surface can further include a low-friction coating or micro-texturing to reduce resistance during manual or ultrasound-assisted lymphatic drainage, and in some embodiments, the surface can be modular or interchangeable to accommodate different patient anatomies or treatment zones. By facilitating uniform distribution of mechanical and/or acoustic energy across the skin, the contoured massage surface supports both manual and ultrasound-enhanced lymphatic stimulation protocols as described in the invention. This design addresses the technical challenge of delivering effective lymphatic drainage without inducing tissue trauma or inconsistent energy application, thereby improving the safety, comfort, and reproducibility of postoperative lymphatic therapy.

### Pressure Sensor Array

The pressure sensor array can comprise a plurality of discrete sensing elements, such as capacitive or piezoresistive cells, arranged in a spatially distributed configuration to detect and quantify the magnitude and distribution of externally applied pressure across a defined surface area. The array can generate real-time, spatially resolved pressure data, with sampling frequencies of at least 10 Hz, thereby capturing dynamic changes in contact force during manual lymphatic drainage or therapeutic ultrasound application. Each sensor element can detect pressure within a range of 0.1 to 10N/cm² , enabling the system to distinguish between light, moderate, and firm contact forces. The pressure sensor array can be integrated into the contact surface of an ultrasound transducer, a manual massage applicator, or a wearable patch, facilitating continuous monitoring of applied pressure during lymphatic stimulation procedures. The output of the pressure sensor array can be transmitted to a control unit, which utilizes the data to provide closed-loop feedback, such as adjusting ultrasound intensity, modulating massage force, or alerting the operator to deviations from a predefined pressure profile. The array can be fabricated using flexible substrates to conform to curved anatomical surfaces, and individual sensor calibration may be performed to ensure measurement accuracy. In some implementations, the array supports mapping of pressure vectors to guide optimal lymphatic flow directionality, thereby enhancing the efficacy and reproducibility of the lymphatic drainage protocol. By enabling precise, real-time monitoring and feedback of applied pressure, the pressure sensor array addresses the technical challenge of standardizing and optimizing manual and ultrasound-assisted lymphatic drainage, reducing operator variability, and improving clinical outcomes in postoperative care.

### Control & Feedback Module

The control & feedback module can serve as the central coordination and regulation component within the Ultrasound Lymphatic Treatment System. The control & feedback module can include an embedded microcontroller programmed to set and dynamically adjust ultrasound emission parameters, such as frequency (typically 0.5-3 MHz), pulse duration, duty cycle, and output intensity, in accordance with either pre-established or operator-modifiable treatment protocols. The control & feedback module can also sequence the timing and duration of manual lymphatic drainage massage cycles, providing operator prompts or synchronizing with automated actuators when present. To ensure safe and effective operation, the control & feedback module can incorporate safety interlocks that monitor system status, including transducer-skin contact, applicator temperature, and patient movement, and can interrupt or modify treatment delivery in response to detected anomalies. The control & feedback module can continuously record treatment session data-including parameter settings, session duration, and event logs-in onboard memory, supporting traceability and compliance. Wireless communication interfaces, such as Bluetooth Low Energy (BLE) and Wi-Fi, can be included to enable secure upload of session data to external devices or cloud-based platforms for postoperative monitoring, compliance tracking, and remote adjustment of treatment regimens. The control & feedback module can further support real-time feedback to the operator or patient via visual, auditory, or haptic indicators, thereby facilitating adherence to the prescribed protocol and enhancing treatment reproducibility. In alternative implementations, the control & feedback module can be integrated with hospital electronic medical record (EMR) systems or mobile health applications to support comprehensive postoperative care management. By providing centralized, adaptive, and safety-focused control, the control & feedback module addresses the technical challenges of standardizing and optimizing lymphatic therapy delivery, ensuring both protocol compliance and patient safety in diverse clinical and home-care environments.

### User Interface Panel

The user interface panel can include a 3.5-inch capacitive touchscreen display integrated with a set of status light-emitting diodes (LEDs), forming the primary operator interface for the Ultrasound Lymphatic Treatment System. The touchscreen can provide a graphical user interface (GUI) that enables clinicians to select and customize treatment protocols, adjust ultrasound parameters such as frequency, intensity, and session duration, and initiate or pause therapy sessions as required by the clinical workflow. The GUI can display real-time feedback on treatment progress, including elapsed time, current ultrasound output, and recommended applicator movement patterns, thereby supporting adherence to the prescribed lymphatic drainage protocol. The status LEDs can provide immediate visual indicators of device operational states, such as power status, readiness for use, active treatment, and error or alert conditions, allowing for rapid recognition of system status even when the touchscreen is not actively monitored. The user interface panel can further support user authentication, session logging, and context-sensitive help prompts, ensuring that only authorized personnel can modify treatment parameters and that all session data are recorded for compliance and quality assurance. The panel can be mounted on the main device housing or connected via a flexible cable, allowing ergonomic positioning relative to the patient and operator. By integrating these features, the user interface panel addresses the technical challenge of standardizing and simplifying the operation of complex lymphatic therapy protocols, reducing user error, and ensuring that clinicians are guided through each procedural step with live metrics relevant to lymphatic stimulation and patient safety.

### Data Logging Memory

The data logging memory can comprise a non-volatile flash storage component, typically with a capacity ranging from 4 GB to 32 GB, and is integrated within the Control & Feedback Module of the Ultrasound Lymphatic Treatment System. The data logging memory can be configured to record and retain comprehensive data from up to 500 individual treatment sessions, including time-stamped sensor traces such as ultrasound transducer output, applicator position, treatment duration, and patient-specific parameters. The data logging memory can store both raw and processed sensor signals, operator input logs, and system status events, thereby enabling detailed documentation of each therapy session for medical record-keeping, regulatory compliance, and retrospective analysis. The memory can support secure data access and transfer, allowing authorized retrieval of session histories for clinical review, outcome tracking, or integration with electronic health record (EHR) systems. In one implementation, the data logging memory can be realized as an embedded flash chip, a removable memory card, or an encrypted solid-state drive, and can be configured to support data overwrite protocols, session indexing, and error correction to ensure data integrity over repeated use. By providing robust, secure, and high-capacity storage of session data, the data logging memory addresses the technical challenge of ensuring traceable, auditable, and interoperable documentation of lymphatic therapy, which is essential for clinical validation, regulatory reporting, and optimization of postoperative care protocols.

### Connectivity Interface

The connectivity interface can include integrated wireless communication modules, such as Bluetooth Low Energy (BLE) 5.0 and IEEE 802.11n (Wi-Fi) radios, that enable bidirectional data exchange between the ultrasound lymphatic treatment system and external devices or cloud-based platforms. The connectivity interface can support remote transmission and reception of device configuration parameters, including ultrasound frequency, pulse duration, and treatment protocol updates, which may be delivered via secure over-the-air (OTA) firmware or software updates. In addition, the connectivity interface can facilitate real-time or periodic upload of treatment session data, such as device usage logs, patient-specific therapy parameters, and outcome metrics, to remote servers for analytics and longitudinal tracking. The connectivity interface can further support encrypted communication protocols, such as AES-256 or TLS 1.3, to ensure patient data privacy and system integrity. In some implementations, the connectivity interface can enable integration with electronic health record (EHR) systems or clinician dashboards, allowing remote monitoring, adjustment of therapy regimens, and aggregation of outcome data for research or quality improvement purposes. The wireless modules of the connectivity interface can be selected and configured to minimize electromagnetic interference with therapeutic ultrasound operation and to comply with relevant medical device communication standards. By providing secure, standards-compliant, and low-interference wireless connectivity, the connectivity interface addresses the technical challenge of enabling remote protocol management, real-time data reporting, and integration with clinical information systems, all while maintaining the safety and efficacy of ultrasound-based lymphatic therapy.

### Patient Positioning & Applicator Mount

The patient positioning & applicator mount can include an adjustable harness, strap, or support assembly that secures one or more therapeutic ultrasound transducers in a fixed spatial relationship to the patient's body. The system can maintain consistent transducer-to-skin contact, pressure, and orientation throughout the entire duration of lymphatic drainage therapy sessions, including during extended treatments or when the patient is ambulatory. The harness can incorporate modular attachment points, adjustable tensioning mechanisms, and articulated joints or pivoting mounts, which allow the system to accommodate anatomical variability and enable precise alignment of the transducer with targeted lymphatic pathways or nodal basins. The mounting system can be fabricated from biocompatible, sterilizable materials and may include padding or conformable surfaces to distribute pressure evenly and minimize patient discomfort. In some implementations, the system can integrate sensors to monitor contact force, angle, or applicator position, providing real-time feedback to ensure protocol adherence and reproducibility. The mounting assembly can be adapted for use on various body regions, including the abdomen, limbs, neck, or axilla, and can be configured for single or multiple simultaneous transducer placements. By enabling hands-free operation of the ultrasound applicator, the patient positioning & applicator mount facilitates standardized, repeatable delivery of therapeutic energy and allows for concurrent manual lymphatic drainage by a clinician or self-administration by the patient. This approach addresses the technical challenge of maintaining consistent and reproducible applicator placement and pressure, which is essential for effective and safe lymphatic stimulation, and overcomes limitations of prior art that relied on manual, operator-dependent positioning.

### Sensor Suite

The sensor suite can comprise an integrated array of biosensors configured to monitor a range of physiological and mechanical parameters of tissue during the application of manual lymphatic drainage and therapeutic ultrasound. The sensor suite can include, but is not limited to, pressure sensors, skin impedance sensors, temperature sensors, and ultrasound backscatter detectors. These sensors can be positioned in proximity to the treatment site, either embedded within the ultrasound applicator, incorporated into wearable patches, or affixed directly to the skin surface. The sensor suite is operatively connected to a control module, which can receive real-time data regarding tissue compliance, interstitial fluid displacement, local temperature changes, and acoustic impedance variations. Based on these measurements, the system can dynamically adjust ultrasound intensity, frequency, duty cycle, and the magnitude or pattern of manual massage pressure to optimize lymphatic flow and minimize tissue trauma. In certain implementations, the sensor suite can further include photoplethysmography or near-infrared spectroscopy elements to assess microvascular perfusion and lymphatic vessel patency. The suite is designed to enable closed-loop feedback control, wherein sensor outputs are continuously analyzed to modulate therapeutic parameters in response to tissue response profiles. This configuration allows for individualized treatment regimens that account for patient-specific anatomical and physiological variability, thereby enhancing the efficacy and safety of the lymphatic drainage protocol. The sensor suite can be calibrated to detect threshold values for edema, tissue elasticity, or lymphatic vessel distension, triggering automated adjustments or operator alerts as needed. In alternative embodiments, the sensor suite may utilize wireless communication to transmit sensor data to a remote monitoring system for further analysis or documentation. By integrating these sensing modalities, the sensor suite addresses the technical challenge of providing real-time, patient-specific feedback that enables adaptive, safe, and effective lymphatic therapy-capabilities not present in conventional postoperative care systems.

### Tissue Impedance Sensor

Generally, the sensor suite can include a tissue impedance sensor configured to measure electrical impedance across skin and subcutaneous tissue at a treatment site. The tissue impedance sensor can comprise a set of electrodes (e.g., four-terminal electrodes) arranged circumferentially around the transducer applicator and/or the massage implement. In one implementation, the tissue impedance sensor applies a low-amplitude alternating current between two drive electrodes while measuring resulting voltage across two sense electrodes, thereby enabling four-point impedance measurement that reduces influence of electrode-skin contact resistance. The tissue impedance sensor can operate over a frequency range (e.g., between approximately 1 kHz and approximately 1 MHz) to differentiate between extracellular and intracellular fluid compartments. More specifically, the tissue impedance sensor can sample impedance values continuously and/or at defined intervals before, during, and/or after ultrasound application and/or manual lymphatic drainage. Shifts in measured impedance can be interpreted as correlates of interstitial fluid movement, with decreases in impedance typically indicating increased fluid mobilization and/or lymphatic drainage. The control and feedback module can process impedance data from the tissue impedance sensor to provide real-time feedback to an operator, such as visual and/or auditory cues indicating sufficient lymphatic activation and/or a need to adjust treatment parameters. Additionally or alternatively, impedance sensor data can be logged in the data logging memory for post-treatment analysis and/or used to automatically modulate ultrasound output parameters of the ultrasound delivery subsystem to optimize lymphatic stimulation.

### Contact Pressure Sensor

Generally, the sensor suite can include a contact pressure sensor positioned beneath the transducer applicator. The contact pressure sensor can comprise a thin-film force-sensing element configured to measure the magnitude of coupling force exerted between the transducer applicator and the skin of a patient during lymphatic drainage procedures. In one implementation, the contact pressure sensor can detect applied forces within a calibrated range (e.g., between approximately 2 and 6 newtons), which can correspond to an optimal pressure window for effective acoustic energy transmission while reducing tissue trauma and/or avoiding compromise of lymphatic flow. The contact pressure sensor can utilize piezoresistive, capacitive, and/or resistive thin-film technology to provide real-time feedback to an operator and/or to the control & feedback module. Additionally, output from the contact pressure sensor can trigger visual and/or auditory alerts when the applied force deviates from a predetermined range, thereby facilitating treatment consistency and/or reducing operator-dependent variability. In one embodiment, the contact pressure sensor can be implemented as a discrete element affixed to the housing of the transducer applicator and/or as an integrated layer within the transducer-skin interface. The contact pressure sensor can also be configured to log force data for post-procedure analysis and/or quality assurance purposes.

### Skin Temperature Sensor

Generally, the skin temperature sensor can comprise a non-contact infrared thermopile configured to continuously monitor epidermal temperature of a treatment area during lymphatic drainage procedures. The skin temperature sensor can detect infrared radiation emitted by skin and convert the radiation into an electrical signal proportional to surface temperature, typically with an accuracy within a threshold range (e.g., ±0.2° C.) over a measurement range (e.g., 25° C. to 45° C.). In one implementation, the skin temperature sensor can be integrated into a housing of the transducer applicator, and/or the skin temperature sensor can be positioned adjacent to the skin surface via an external probe. The skin temperature sensor can sample temperature output at configurable intervals (e.g., 0.5 to 2 seconds) and transmit temperature data to the control and feedback module, which can trigger an alert and/or automatically modulate ultrasound power delivery if measured temperature exceeds a predetermined safety threshold (e.g., 39° C.) to prevent thermal injury and/or patient discomfort. Additionally, the skin temperature sensor can log temperature data for post-treatment analysis and/or quality assurance. In some implementations, the skin temperature sensor can be calibrated to account for skin emissivity and/or ambient temperature fluctuations, thereby supporting reliable operation across diverse patient populations and/or anatomical sites.

### Sterile Coupling Medium Cartridge

Generally, the ultrasound lymphatic treatment system can include a sterile coupling medium cartridge configured to facilitate acoustic energy transfer from the transducer applicator to the patient's skin. The sterile coupling medium cartridge can comprise a disposable, single-use housing pre-filled with a predetermined volume (e.g., 10-50 mL) of medical-grade acoustic coupling gel. The housing of the cartridge can include a snap-in and/or twist-lock interface that enables tool-free attachment and detachment from the transducer applicator, thereby reducing operator contact with the gel and/or minimizing cross-contamination risk between patients. In some implementations, the sterile coupling medium cartridge can incorporate a tamper-evident seal and/or a breakable membrane configured to be breached upon installation, ensuring that the coupling medium remains uncontaminated until the point of use. The gel formulation within the cartridge can be optimized for acoustic impedance matching within a therapeutic frequency range (e.g., 0.5-3.0 MHz), supporting efficient transmission of ultrasound energy to subcutaneous lymphatic structures. Additionally, the geometry of the cartridge can be contoured to conform to the applicator head of the transducer applicator, thereby providing uniform gel distribution and/or consistent acoustic coupling across the treatment area. In one embodiment, the cartridge can include an integrated indicator (e.g., a color change element and/or a mechanical flag) configured to signal complete deployment and/or exhaustion of the coupling medium. Thus, the sterile coupling medium cartridge can standardize the coupling process, reduce preparation time, and/or support compliance with infection control protocols during postoperative lymphatic therapy procedures.

### Power Supply Module

Generally, the ultrasound lymphatic treatment system can include a power supply module configured to deliver regulated direct current electrical power to the various components of the system. The power supply module can support both untethered and tethered operation, enabling use in clinical and/or home settings. More specifically, the power supply module can incorporate circuitry for voltage regulation, overcurrent protection, and/or battery management, including charging and discharging control. The power supply module can provide output in a suitable voltage range (e.g., between approximately 5 V DC and approximately 24 V DC) with current capacity sufficient to sustain continuous operation of the ultrasound delivery subsystem, control and feedback module, and/or sensor suite during extended treatment sessions typical of postoperative lymphatic care.

### Manual and Ultrasound-enhanced Lymphatic Drainage Method

The manual and ultrasound-enhanced lymphatic drainage method can comprise a multi-phase therapeutic protocol that integrates manual lymphatic drainage (MLD) techniques with the application of low-frequency therapeutic ultrasound to stimulate and accelerate lymphatic transport in both preoperative and postoperative clinical settings. The method can commence with individualized mapping of the patient's lymphatic anatomy, which can be performed visually, by palpation, or using imaging modalities to identify primary lymphatic vessels, nodal basins, and regions of anticipated fluid accumulation. This mapping step can ensure that subsequent interventions are anatomically targeted and patient-specific, thereby improving the precision and efficacy of the protocol.

Following mapping, the method can proceed with preparatory node clearing, wherein the practitioner applies gentle, repetitive manual strokes to major lymph node clusters, such as the cervical, axillary, and inguinal regions. These strokes can be executed with controlled pressure below 40 mmHg to facilitate downstream lymph flow without collapsing superficial capillaries. The preparatory node clearing can prime the lymphatic system for enhanced fluid mobilization during subsequent steps, addressing the technical challenge of overcoming nodal bottlenecks that impede effective drainage after surgery.

Sequential manual drainage strokes can then be performed in a distal-to-proximal direction along the mapped lymphatic pathways. The practitioner can employ standardized parameters for pressure (typically 20-40 mmHg), stroke length (5-10 cm per stroke), and frequency (10-20 strokes per region) to mobilize interstitial fluid toward the nodal basins. This standardization can reduce operator-dependent variability and ensure reproducible outcomes across different practitioners and sessions. The manual drainage phase can be adapted to the anatomical region and patient tolerance, providing flexibility while maintaining protocol integrity.

After manual drainage, the method can include the application of therapeutic ultrasound using a transducer set to a defined frequency range, such as 0.8-3.0 MHz, and intensity, such as 0.2-1.0 W/cm². The ultrasound applicator can be moved in slow, overlapping passes along the same lymphatic trajectories established during manual mapping and drainage. Ultrasound application parameters—including duty cycle, duration per region, and total session time—can be selected based on patient-specific factors such as tissue thickness, surgical site, and degree of edema. This phase can further enhance lymphatic transport by inducing acoustic micro-vibration and streaming, which can open lymphatic endothelial junctions and promote fluid movement beyond what is achievable with manual techniques alone.

The method can alternate manual and ultrasound modalities in timed cycles, for example, 5-10 minutes per modality per anatomical region. The sequence and number of cycles can be adjusted according to intra-session assessment of tissue response, such as reduction in pitting edema, improvement in skin turgor, or changes in circumference measurements. This adaptive approach can ensure that the protocol remains responsive to real-time patient needs, optimizing therapeutic benefit while minimizing the risk of overtreatment or tissue fatigue.

At the conclusion of each session, the method can include objective outcome measurements, such as volumetric limb assessment, ultrasonographic evaluation of fluid pockets, or photographic documentation of treated regions. These measurements can be used to evaluate the efficacy of the intervention, guide the scheduling of follow-up treatments, and support longitudinal tracking of patient recovery. The protocol can be further adapted for various surgical procedures and anatomical regions by modifying mapping, manual, and ultrasound parameters to suit the specific lymphatic architecture and clinical objectives.

By standardizing the integration of manual and ultrasound-based lymphatic stimulation, the manual and ultrasound-enhanced lymphatic drainage method can provide reproducible parameters for clinical implementation and enable consistent postoperative outcomes, including accelerated edema resolution, reduced ecchymosis, and enhanced immune and metabolic recovery. This method addresses the technical challenges of insufficient postoperative lymphatic care, lack of standardized protocols for lymphatic activation, and the need for reproducible, operator-independent outcomes in lymphatic therapy.

### Patient Evaluation and Lymphatic Zone Mapping

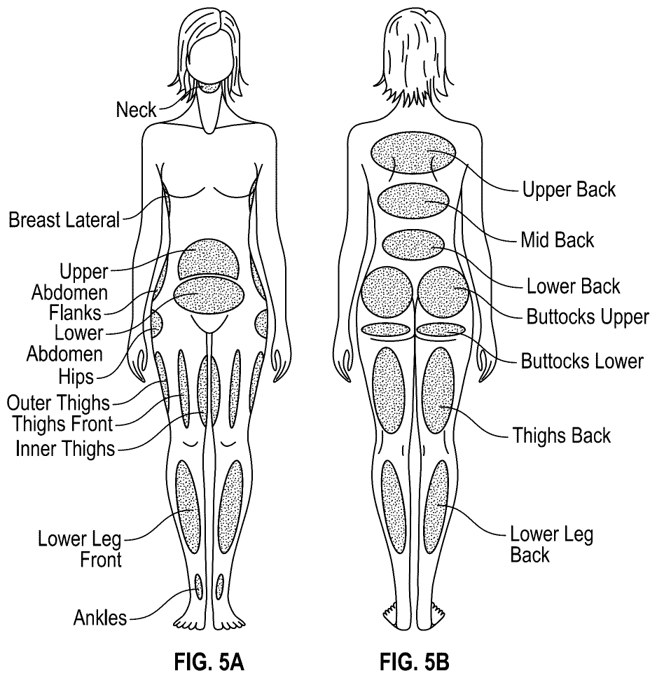

FIG. 5A and FIG. 5B provide schematic references for patient evaluation and lymphatic zone mapping step. The practitioner can begin the evaluation by reviewing the surgical records of the patient to identify the type and anatomical location of any recent procedures, which enables the practitioner to anticipate regions at risk for lymphatic disruption or congestion. The practitioner can then visually inspect the skin for evidence of recent incisions, edema, ecchymosis, or other signs of impaired lymphatic drainage, and can perform palpation over major lymphatic nodal basins—including the cervical, axillary, inguinal, and popliteal regions—to detect areas of induration, tenderness, or reduced compliance that may indicate impaired lymphatic flow. Using a skin-safe marker or a digital mapping device, the practitioner can trace the course of primary superficial and deep lymphatic vessels on the patient's skin, referencing reproducible anatomical landmarks such as the clavicle, costal margin, umbilicus, and inguinal crease. The practitioner can mark the locations of major nodal clusters and delineate drainage territories that correspond to the surgical field and adjacent tissue planes. This mapping process can establish a visual and spatial guide for subsequent manual lymphatic drainage strokes and therapeutic ultrasound sweeps, ensuring that all interventions follow anatomically validated lymphatic drainage vectors. The mapping may be documented photographically or digitally for reproducibility and treatment planning, and can be repeated at each treatment session to account for changes in tissue status or lymphatic flow dynamics. By standardizing the identification and marking of lymphatic pathways and nodal basins, the patient evaluation and lymphatic zone mapping step addresses the technical challenge of operator-dependent variability and ensures that both manual and ultrasound-based interventions are anatomically targeted, reproducible, and optimized for each patient's unique postoperative anatomy.

### Proximal Node Clearance

The step of proximal node clearance can comprise the manual stimulation of each major lymphatic nodal basin to facilitate lymphatic drainage from distal tissues. In one implementation, the practitioner can apply a series of 5 to 10 manual pumping maneuvers to each nodal basin, including but not limited to the cervical, axillary, and inguinal regions. The technique can involve gentle, circular hand motions and stationary compressive holds, with the practitioner maintaining applied pressure below 40 mm Hg to prevent capillary collapse while promoting the opening of lymphatic valves. This sub-threshold pressure regime can generate a transient negative pressure gradient within the lymphatic vessels, thereby enhancing the intrinsic contractility of lymphangions and promoting unidirectional lymph flow toward the central venous system. The step can be performed using either one or both hands, with the practitioner modulating the frequency and duration of compressions based on patient tolerance and anatomical considerations. The method can be adapted to target specific nodal basins relevant to the surgical site or anticipated regions of lymphatic congestion. In some implementations, the step can precede or be combined with adjunctive modalities, such as low-frequency therapeutic ultrasound, to further augment lymphatic valve opening and fluid mobilization. The practitioner can use objective measures, such as tissue compliance or ultrasonographic assessment, to determine the adequacy of nodal clearance before proceeding to subsequent steps in the protocol. By standardizing the approach to proximal node clearance, the invention addresses the technical challenge of ensuring that lymphatic outflow pathways are patent prior to mobilizing distal interstitial fluid, thereby reducing the risk of postoperative edema, seroma formation, and delayed immune recovery.

### Ultrasound Parameter Configuration

The ultrasound parameter configuration step can enable the operator to tailor the therapeutic ultrasound device for optimal lymphatic stimulation while minimizing the risk of tissue trauma. In this step, the operator can select an ultrasound frequency within the range of 0.5 to 3 megahertz (MHz), which allows the acoustic energy to be targeted to the appropriate tissue depth for the lymphatic structures of interest. The operator can also set a duty cycle between 20% and 50%, thereby controlling the proportion of time the ultrasound is actively emitting energy during each pulse cycle. This adjustment can help balance the mechanical stimulation of lymphatic vessels with the need to limit cumulative thermal exposure to the tissue. Additionally, the spatial average temporal-average (SATA) intensity can be adjusted to a value not exceeding 0.8 watts per square centimeter (W/cm²), ensuring that the delivered energy remains within safe and effective therapeutic limits for postoperative lymphatic activation.

Prior to ultrasound application, the operator can apply a water-based coupling gel to the treatment area to facilitate efficient acoustic transmission from the transducer to the skin. In some implementations, the coupling gel can be formulated to include lymphotropic essential oils, such as those containing terpenes or monoterpenoids, which may enhance percutaneous absorption and further augment lymphatic activation through biochemical pathways. The configuration step can be performed according to a standardized protocol or can be tailored to patient-specific factors, such as tissue thickness, surgical site, or the degree of postoperative edema, allowing for individualized therapy that addresses anatomical and clinical variability.

The operator can verify device calibration and ensure that all safety interlocks are engaged prior to initiating ultrasound delivery. This may include running a built-in calibration routine to confirm that the piezoelectric output matches factory specifications and checking that safety features-such as temperature, pressure, and contact sensors-are functioning correctly. In some implementations, the configuration step can further include selecting a transducer head size appropriate for the anatomical region to be treated, as well as programming the device for either pulsed or continuous wave operation depending on the desired depth of penetration and tissue response. All configuration parameters can be recorded in the patient's treatment log, supporting reproducibility of therapy and facilitating outcome tracking across multiple sessions and operators.

By providing a structured and customizable approach to ultrasound parameter configuration, the method addresses the technical challenge of standardizing lymphatic stimulation protocols while allowing for patient-specific adaptation. This ensures that each treatment session delivers a reproducible and evidence-based acoustic dose, supporting improved postoperative outcomes and enabling consistent documentation for clinical research and quality assurance.

### Frequency Selection

Generally, frequency selection can comprise determining and/or applying an ultrasound frequency appropriate for an anatomical depth of target lymphatic structures. More specifically, the ultrasound delivery subsystem can select an ultrasound frequency that produces acoustic streaming and/or mechanical stimulation at a desired tissue depth to facilitate lymphatic flow. For deeper anatomical regions (e.g., the trunk and/or thigh), the ultrasound delivery subsystem can utilize an ultrasound frequency in a range (e.g., approximately 0.5 to 1 MHz), which can achieve a penetration depth (e.g., approximately 3 to 4 centimeters), thereby targeting deep lymphatic vessels and/or nodal basins. Alternatively, for more superficial anatomical regions (e.g., the face and/or breast), the ultrasound delivery subsystem can employ a higher ultrasound frequency in a range (e.g., approximately 2 to 3 MHz), which can result in a shallower penetration depth (e.g., approximately 1 to 2 centimeters), suitable for stimulating superficial lymphatic channels and/or capillaries. In one implementation, the frequency selection can be based on preoperative imaging, anatomical landmarks, and/or standardized treatment protocols. Additionally, an operator can adjust the frequency in real time according to patient-specific tissue thickness and/or a location of surgical intervention. In one embodiment, the ultrasound delivery subsystem can output an ultrasound parameter set comprising the selected frequency and/or corresponding anatomical region to ensure reproducibility and/or protocol adherence.

### Duty Cycle and Intensity Setting

Generally, the ultrasound delivery subsystem can operate in a pulsed emission mode during duty cycle and intensity setting. The duty cycle can be set to a target percentage (e.g., between approximately 10% and 30%) to reduce the average thermal load delivered to tissue while maintaining sufficient mechanical micro-vibration to facilitate transient opening of lymphatic endothelial cell junctions. In one implementation, the ultrasound delivery subsystem incrementally increases an ultrasound intensity in discrete steps (e.g., approximately 0.05 to 0.2 W/cm² increments) until a patient perceives mild tissue warming. The intensity can be maintained below a threshold that would induce visible erythema and/or discomfort. Alternatively, the duty cycle and/or intensity can be adjusted based on patient feedback, anatomical site, and/or clinical objectives to optimize lymphatic endothelial response while minimizing risk of thermal injury. In the breast-centric postoperative protocol variant, the duty cycle and intensity setting can be configured for sensitive postoperative tissue, and the ultrasound delivery subsystem can apply more conservative intensity increments. The duty cycle and intensity setting can output an ultrasound parameter set and can result in the ultrasound system parameterised configuration, thereby enabling subsequent static nodal activation and/or dynamic vessel sweeping.

### Coupling Medium Application

Generally, coupling medium application can include applying a coupling medium to a designated treatment zone prior to the administration of therapeutic ultrasound. The coupling medium can be a gel, hydrogel sheet, and/or other acoustically transparent material configured to facilitate efficient transmission of ultrasonic energy from the transducer applicator to underlying tissue. In one implementation, the coupling medium can be distributed to form a continuous layer with a thickness within an exemplary range (e.g., approximately 1 to 2 millimeters), although the thickness can be adjusted based on anatomical contour, transducer type, and/or specific treatment requirements. The coupling medium can function to eliminate air interfaces between the transducer applicator and the skin, thereby minimizing acoustic reflection losses and/or maximizing energy transfer. Additionally, the coupling medium can enable smooth, uniform gliding of the transducer applicator across the skin surface, reducing risk of mechanical irritation and/or uneven energy delivery. In some implementations, the coupling medium can incorporate additives such as humectants, anti-inflammatory agents, and/or antimicrobial compounds to further support tissue recovery and/or reduce infection risk. The sterile coupling medium cartridge can execute coupling medium application immediately prior to each ultrasound application cycle and/or can repeat application as needed to maintain optimal acoustic coupling throughout a treatment session. Thus, coupling medium application can produce an applied coupling medium layer that serves as an input to subsequent static nodal activation and/or dynamic vessel sweeping.

### Distal-to-Proximal Manual Drainage Sequence

The distal-to-proximal manual drainage sequence can comprise a series of rhythmic, skin-stretching strokes performed by a therapist or operator along predetermined lymphatic pathways, beginning at the most distal anatomical region—such as the hands, feet, or lower extremities-and progressing incrementally toward the proximal regions of the torso. The method can specify that each stroke is applied with sufficient pressure to mobilize interstitial fluid within the superficial lymphatic vessels, while remaining below the threshold that would cause discomfort or tissue trauma. In one implementation, each stroke can be executed over a duration of approximately 1 to 2 seconds and positioned such that it overlaps the preceding stroke by approximately 50%, thereby maintaining a continuous columnar movement of fluid toward central lymphatic basins. The sequence can be performed in conjunction with mapped anatomical landmarks, as defined by preoperative or intraoperative assessment, to ensure precise alignment with major lymphatic conduits and nodal clusters. The technique can be adapted to various body regions, including but not limited to the limbs, trunk, and cervical areas, and may be repeated in multiple passes to optimize lymphatic clearance. In another implementation, the sequence can be performed manually or in combination with adjunctive modalities, such as low-frequency therapeutic ultrasound, to further enhance lymphatic flow. The method can specify the directionality, stroke length, frequency, and overlap percentage to standardize the procedure and facilitate reproducibility across operators and patient anatomies. By defining these parameters, the distal-to-proximal manual drainage sequence addresses the technical challenge of standardizing and optimizing lymphatic fluid mobilization, thereby improving the efficacy and reproducibility of postoperative lymphatic care compared to prior art that lacks such protocolized directionality and overlap control.

### Upper Body Manual Stroke Sequence

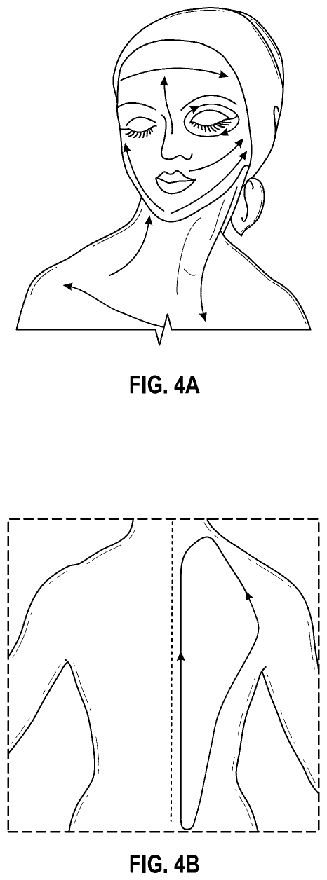

(FIG. 4A) The upper body manual stroke sequence can include a series of manual lymphatic drainage maneuvers applied to the upper extremities and anterior thorax, with the objective of augmenting superficial lymphatic flow and accelerating the clearance of postoperative exudate. The practitioner can initiate the sequence at the most distal point of the treated segment—such as the fingertips, dorsal hand, or inferolateral breast border—and direct each manual stroke proximally toward a major lymph node basin, typically the axillary or supraclavicular clusters. The operator can apply gentle, rhythmic pressure using the palmar surface of the hand, maintaining a unidirectional trajectory that mirrors the anatomical course of superficial lymphatic vessels as visualized in standard anatomical references and as depicted by the curvilinear markings in FIG. 4A. The technique can incorporate both linear and circular motions, with the choice of pattern determined by local tissue compliance and the orientation of lymphatic collectors; for example, linear strokes may be favored along the forearm, while semicircular or figure-of-eight patterns can be employed over the deltoid or lateral breast mound to optimize lymphatic valve opening and fluid mobilization. When recent surgical incisions are present within the treatment field, the practitioner can activate an incision-bypass protocol, detouring stroke trajectories at least 2 cm lateral to the wound margin and funneling interstitial fluid toward intact adjacent tissue planes, thereby protecting wound integrity while still mobilizing fluid from the sub-incisional lymphatic plexus. The sequence can be adapted in stroke length, pressure, and frequency based on patient tolerance, tissue compliance, and the degree of postoperative edema, with the applied surface pressure maintained within a therapeutic band of 20-40 mm Hg, verified by tactile manometer or wearable pressure-sensing glove. The protocol can specify a minimum number of passes-typically 5 to 10 per region-and a standardized duration of 2 to 5 minutes per treatment zone, with cadence and pass count tracked using a digital metronome or tally device to ensure reproducibility. The sequence can be performed as a standalone intervention or in conjunction with therapeutic ultrasound application, with the manual phase preceding, following, or alternating with acoustic stimulation in micro-cycles to maximize synergistic lymph propulsion. By codifying stroke direction, pressure, pass count, and timing, the protocol standardizes therapeutic dose and enables reproducible, operator-independent delivery of mechanical lymphatic stimulation, addressing the technical challenge of inconsistent postoperative lymphatic care and supporting improved edema resolution, reduced bruising, and enhanced patient comfort.

### Lower Body Manual Stroke Sequence

The lower body manual stroke sequence can include a series of manual lymphatic drainage strokes applied to the lower extremities, with the primary objective of mobilizing interstitial fluid toward the inguinal lymph nodes. The method can initiate at the most distal regions of the lower limb, such as the dorsal ankle or the outer aspect of the thigh, and progress proximally along the anatomical course of the superficial and deep lymphatic vessels, specifically following the saphenous and femoral lymphatic pathways as depicted in FIG. 4B. The therapist can position the patient's limb at an elevation angle of approximately five degrees relative to the horizontal plane, thereby leveraging gravity-assisted lymphatic return to supplement manual propulsion. Each stroke can be performed using gentle, rhythmic, and unidirectional manual pressure, with the direction of each pass oriented strictly toward the inguinal nodal basin to maintain valve patency and prevent retrograde flow. The sequence can be executed either independently or in conjunction with concurrent therapeutic ultrasound application, with the timing of manual strokes coordinated to follow acoustic passes when both modalities are used. The duration, frequency, and pressure of each stroke can be modulated in real time based on patient-specific factors such as tissue compliance, degree of postoperative edema, and individual tolerance, with the protocol specifying a defined number of repetitions per limb segment and incorporating subsidiary redirect strokes from lateral or posterior compartments as needed. The sequence can be adapted for unilateral or bilateral limb treatment and may be integrated into a comprehensive postoperative lymphatic therapy regimen to accelerate interstitial fluid clearance, reduce edema, and promote metabolic and immune recovery. By standardizing the directionality, pressure, and sequence of manual strokes, the lower body manual stroke sequence addresses the technical challenge of operator-dependent variability and ensures reproducible, anatomically targeted lymphatic drainage, which is not achieved by conventional postoperative care protocols.

### Ultrasound Application Along Lymphatic Pathways

The step of ultrasound application along lymphatic pathways can include positioning a therapeutic ultrasound transducer, having a contact surface diameter between 2 cm and 5 cm, in direct contact with the patient's skin along anatomically mapped lymphatic drainage routes. In one implementation, the operator can move the transducer in a continuous, linear or curvilinear motion that follows distal-to-proximal trajectories, as visually depicted in FIG. 4A, which illustrates preferred sweep directions and anatomical vectors for both manual and ultrasound-based lymphatic stimulation. The movement speed of the transducer can be maintained within a range of 2 to 4 centimeters per second, a parameter selected to ensure consistent acoustic energy delivery to the subcutaneous tissue while minimizing the risk of excessive tissue heating or uneven stimulation. At each anatomically significant lymph node cluster-such as the cervical, axillary, inguinal, or popliteal regions-the operator can pause the transducer in a static position for a dwell time of 30 to 60 seconds. This static application is intended to locally stimulate lymphatic vessel peristalsis and enhance nodal fluid mobilization, leveraging the mechanical and acoustic effects of low-frequency ultrasound to augment lymphatic throughput.

The ultrasound frequency, intensity, and duty cycle can be selected based on the desired depth of penetration and the tissue response required for the specific anatomical region. In one example, frequencies in the range of 0.5 to 3 MHz and intensities from 0.1 to 1.5 W/cm² can be employed, with the system supporting both pulsed and continuous emission modes. The operator or control module can adjust these parameters in real time to match the anatomical depth of the lymphatic vessels and nodal basins, as well as to accommodate patient-specific factors such as tissue compliance, postoperative edema, or surgical site sensitivity. The protocol can be adapted to target specific anatomical regions based on the type of surgery performed and the location of lymphatic congestion, ensuring that the ultrasound application is both anatomically precise and therapeutically effective.

In some implementations, the ultrasound application along lymphatic pathways can be performed in conjunction with, or immediately following, manual lymphatic drainage. This sequencing can synergistically augment lymphatic flow by combining the mechanical effects of manual tissue deformation with the acoustic micro-vibration and streaming generated by the ultrasound transducer. The system can further incorporate real-time feedback from integrated sensors—such as contact pressure, skin temperature, and tissue impedance—to dynamically adjust ultrasound output and ensure that energy delivery remains within safe and effective limits. By standardizing the movement path, speed, and dwell times, and by integrating closed-loop parameter control, the invention addresses the technical challenge of delivering reproducible, operator-independent lymphatic stimulation that accelerates postoperative recovery, reduces interstitial edema, and improves immune and metabolic outcomes compared to conventional manual-only or non-standardized ultrasound protocols.

### Static Nodal Activation

Static Nodal activation can involve positioning the transducer applicator in a stationary manner directly over a targeted lymph node and/or nodal cluster. Generally, the ultrasound delivery subsystem can emit low-frequency ultrasound pulses (e.g., between approximately 0.5 and 3.0 MHz) while the transducer applicator remains stationary relative to the treatment site. More specifically, the stationary application of ultrasound energy can induce localized acoustic micro-streaming and/or cavitational effects within the lymph node, resulting in mechanical agitation of lymph fluid and/or cellular components. In one implementation, the ultrasound delivery subsystem can maintain the stationary transducer position for a treatment duration (e.g., between approximately 30 and 120 seconds per node) at an intensity level (e.g., between approximately 0.2 and 1.5W/cm² ). The ultrasound parameters, including frequency, intensity, and/or pulse repetition rate, can be adjusted based on anatomical location of the targeted node, patient tolerance, and/or desired depth of penetration. In the breast-centric postoperative protocol variant, static nodal activation can target axillary and/or supraclavicular nodal clusters associated with breast lymphatic drainage pathways. Thus, static nodal activation can address the challenge of poor post-surgical immune and metabolic recovery by mechanically stimulating lymph movement through nodal sinuses and potentially enhancing phagocytic activity of resident immune cells.

### Dynamic Vessel Sweeping

Generally, dynamic vessel sweeping can include moving the transducer applicator along mapped lymphatic vessel pathways using elongated, S-shaped trajectories. More specifically, the ultrasound delivery subsystem can position the transducer applicator so that each sweep follows the anatomical course of superficial and/or deep lymphatic vessels as identified by the patient-specific lymphatic zone map. In one implementation, each pass of the transducer applicator can be overlapped with a preceding pass by a coverage ratio (e.g., approximately 50% of a transducer head width), thereby reducing untreated acoustic zones and/or promoting uniform energy delivery across a target vessel region. The S-shaped motion can be executed manually by an operator and/or by a programmable actuator, and sweep velocity, contact pressure, and ultrasound emission parameters of the ultrasound parameter set can be adjusted according to patient tissue characteristics and/or treatment objectives. Additionally, the direction of sweeping can be oriented toward regional lymph node basins to facilitate lymph propulsion, and dynamic vessel sweeping can be applied to various anatomical regions including the abdomen, limbs, breast, and/or neck, with the sweep path tailored to local lymphatic architecture. Thus, dynamic vessel sweeping addresses the absence of protocols combining manual lymphatic massage with therapeutic ultrasound by providing a systematic approach to stimulate lymphatic flow along vessel pathways, thereby enhancing drainage efficiency and/or reducing post-surgical swelling.

### Alternating Manual-Ultrasound Cycles

Generally, the manual and ultrasound-enhanced lymphatic drainage method can include alternating manual-ultrasound cycles in which a practitioner sequentially performs manual lymphatic drainage strokes and therapeutic ultrasound application to the same or adjacent lymphatic drainage basins. In one implementation, each cycle can comprise a manual phase followed by an ultrasound phase, with each phase lasting a target interval (e.g., approximately 5 minutes), and the alternating cycles can continue for a cumulative session duration (e.g., between 30 and 45 minutes). During the manual phase, the practitioner can execute lymphatic drainage techniques-such as effleurage, stationary circles, and/or directional strokes-along predetermined lymphatic pathways using the massage implement of the manual lymphatic drainage subsystem. The subsequent ultrasound phase can involve the control & feedback module directing the ultrasound delivery subsystem to apply low-frequency therapeutic ultrasound to the region treated during the preceding manual phase, with the transducer applicator positioned according to the calibrated ultrasound parameter set. More specifically, the interleaving of manual and ultrasound phases can generate repeated, alternating pressure gradients within interstitial and lymphatic compartments, thereby promoting centripetal lymph propulsion toward nodal basins and central ducts. The ultrasound parameters for each ultrasound phase can be standardized and/or adjusted based on patient-specific factors such as tissue thickness, surgical site location, and/or degree of edema. Alternatively, the duration and/or sequence of the manual and ultrasound cycles can be modulated to accommodate anatomical region, patient tolerance, and/or clinical objectives. In another implementation, the alternation protocol can be implemented as a fixed regimen or as a dynamically adjusted sequence based on real-time assessment of tissue response, lymphatic flow, and/or operator feedback provided through the sensor suite. Thus, the alternating manual-ultrasound cycles can address the absence of protocols combining manual lymphatic massage with therapeutic ultrasound by providing a structured interleaving regimen that prevents localized tissue fatigue, maintains optimal tissue compliance, and enhances the synergistic effects of mechanical and acoustic stimulation for improved post-surgical lymphatic drainage.

### Post-treatment Assessment and Documentation

Generally, the post-treatment assessment and documentation step can comprise a sequence of actions performed immediately following completion of a manual and ultrasound-enhanced lymphatic drainage session. In one implementation, the control and feedback module can collect quantitative and qualitative data to evaluate physiological and/or subjective effects of the therapy. More specifically, the ultrasound lymphatic treatment system can facilitate measurement of anatomical parameters, such as limb circumference and/or abdominal girth, at standardized anatomical landmarks. The control and feedback module can further acquire high-resolution digital photographs of incision sites and/or treated regions, ensuring consistent camera angle, distance, and/or lighting to enable objective comparison across sessions. Additionally, the treatment protocol software can record patient-reported outcomes, such as pain intensity, using validated scales (e.g., a numeric rating scale ranging from 0 to 10 and/or a visual analog scale). All collected data-including measurement values, photographic records, and/or subjective scores-can be entered into the data logging memory and/or transmitted via the connectivity interface to an electronic health record system, thereby enabling longitudinal tracking of treatment efficacy, facilitating comparison between sessions, and/or supporting protocol optimization.

### Edema Quantification

The step of edema quantification can comprise the objective assessment of postoperative tissue swelling by measuring changes in limb or body segment volume before and after execution of the Manual and Ultrasound-Enhanced Lymphatic Drainage Method. In one implementation, the practitioner can perform circumferential measurements using a flexible, non-elastic tape at standardized anatomical landmarks, such as the mid-patella, malleolus, or umbilicus, to ensure reproducibility across sessions and operators. In another implementation, the system can employ three-dimensional (3-D) optical scanning systems that generate volumetric reconstructions of the treated region, allowing for high-resolution, operator-independent quantification of edema. The edema quantification process can include recording a baseline measurement immediately prior to intervention, after the patient has rested supine for at least 10 minutes to allow for fluid redistribution, and subsequent measurements at defined postoperative intervals, such as 24 hours, 72 hours, and 7 days after treatment. The system can automatically calculate the percentage change in volume or circumference, with a reduction of at least 5% relative to baseline considered a clinically meaningful decrease in edema. In some variants, the system can further log all measurement data digitally, correlate changes with patient-reported outcomes or photographic documentation, and export the results to the electronic health record for longitudinal tracking. By standardizing measurement protocols and automating data analysis, the edema quantification step addresses the technical challenge of providing an objective, reproducible endpoint for evaluating the efficacy of manual and ultrasound-enhanced lymphatic drainage interventions, thereby enabling protocol optimization and multi-center comparability.

### Pain and Mobility Scoring

The pain and mobility scoring step can quantitatively and qualitatively assess postoperative pain and functional mobility in a subject undergoing the Manual and Ultrasound-Enhanced Lymphatic Drainage Method. In one implementation, the practitioner can record subjective pain intensity using a visual analog scale (VAS), which allows the subject to indicate perceived pain on a continuous scale from 0 (no pain) to 10 (worst imaginable pain). Functional mobility can be evaluated by performing goniometric measurements of joint angles relevant to the surgical or treated region, thereby documenting the degree of active or passive movement possible. The method can schedule these assessments at predetermined intervals, such as before, immediately after, and at follow-up points post-intervention, to monitor changes in pain and mobility over time. In another implementation, the practitioner can supplement or replace the VAS and goniometry with additional validated pain or function scoring systems, provided these alternatives yield reproducible and quantifiable outcomes suitable for clinical documentation and analysis. By systematically collecting pain and mobility data, the method enables tracking of recovery trajectories, comparison of protocol variants, and informed adjustment of subsequent treatment regimens. This approach addresses the technical challenge of objectively measuring the efficacy of combined manual and ultrasound lymphatic drainage protocols, providing standardized, reproducible outcome metrics that support protocol optimization and regulatory compliance.

### Session Scheduling and Patient Education

The Manual and Ultrasound-Enhanced Lymphatic Drainage Method can include a session scheduling and patient education step that determines and communicates the timing and frequency of follow-up lymphatic therapy sessions based on the patient's postoperative assessment outcomes. The method can establish an initial follow-up interval of 48 to 72 hours after surgery, with subsequent sessions scheduled at weekly intervals until clinical indicators of edema, such as limb or region circumference measurements, reach a stable plateau. The scheduling protocol can be dynamically adjusted according to patient-specific factors, including the extent of surgical intervention, baseline lymphatic function, and the observed response to initial therapy sessions, thereby individualizing the cadence of care to optimize recovery.

In addition to scheduling, the method can instruct the patient in self-administered lymphatic drainage techniques. The practitioner can demonstrate and supervise practice of manual self-massage along prescribed lymphatic pathways, emphasizing correct stroke directionality, pressure parameters (such as maintaining pressure below 40 mm Hg), and repetition count. Educational content can be delivered verbally, in written form, or via digital media, and may include diagrams or video demonstrations tailored to the anatomical region treated, such as the abdomen, breast, or lower limb. The patient can also receive guidelines for maintaining adequate hydration, with specific fluid intake targets provided to support lymphatic flow, and be advised on monitoring for signs of excessive swelling, bruising, or discomfort between sessions. The method can further provide a checklist of warning signs and escalation pathways, instructing the patient on when to seek additional clinical evaluation.

To support adherence and documentation, the method can supply standardized patient education materials and a session log or digital tracking tool for recording self-care activities and symptom progression. In one implementation, the method can incorporate remote follow-up via telemedicine platforms, enabling the patient to upload images or symptom logs and receive reminders or feedback from the clinical team. This approach allows the session schedule to be adjusted in response to patient-reported outcomes, reinforcing self-care adherence and optimizing resource utilization. By integrating adaptive scheduling, comprehensive patient education, and remote monitoring, the method addresses the technical challenge of maintaining continuous, individualized lymphatic stimulation and early complication detection during the postoperative recovery period.

I claim:

1.-9. (canceled)

10. The method of claim 9, wherein applying therapeutic ultrasound further comprises performing static-nodal activation that includes dwelling the ultrasound transducer for 30 seconds to 60 seconds at each cervical, axillary, and inguinal lymph-node clusters.

11. The method of claim 9, wherein the dynamic-vessel sweeping is performed with elongated S-shaped trajectories, each trajectory overlapping a preceding trajectory by approximately 50 percent of a width of the transducer applicator.

12. The method of claim 9, further comprising applying a bubble-free coupling medium as a continuous film having a thickness between 1 millimeter and 2 millimeters to yield a coupling-layer-present-on-skin configuration before each ultrasound phase.

13. The method of claim 9, further comprising executing, 24 hours to 48 hours before surgery, a pre-operative priming regimen that performs steps a) through f) at an acoustic intensity not exceeding 0.15 watts per square centimeter.

14. The method of claim 9, wherein the method is performed in a home-use postoperative variant that employs a wearable belt-mounted ultrasound array and a mobile application configured to issue stroke-cadence prompts during the manual-lymphatic-drainage phases.

15.-17. (canceled)

18. The method of claim 9, further comprising computing edema-trend slopes from the post-treatment outcome dataset and, based on the edema-trend slopes, dynamically shortening or lengthening intervals between subsequent lymphatic-therapy sessions in the follow-up schedule.

19. The method of claim 9, further comprising automatically modulating the duty cycle of the therapeutic ultrasound between 20 percent and 30 percent when the skin-temperature sensor detects that a superficial tissue temperature approaches 39 degrees Celsius.

20. (canceled)
